(19) United States
(12) Patent Application Publication
(10) Pub. No.: US 2005/0075599 A1
(43) Pub. Date: Apr. 7, 2005

(54) Ultrasonically enhanced saline treatment for burn damaged skin

(76) Inventor: Bruce K. Redding JR., Broomall, PA (US)

Correspondence Address: REED SMITH LLP, 2500 ONE LIBERTY PLACE, 1650 MARKET STREET, PHILADELPHIA, PA, 19103, US

(21) Appl. No.: 10/782,398

(22) Filed: Feb. 18, 2004

Related U.S. Application Data
(63) Continuation-in-part of application No. 09/939,435, filed on Aug. 24, 2001, now abandoned.
(63) Continuation-in-part of application No. 09/939,506, filed on Aug. 24, 2001, now abandoned.
(63) Continuation-in-part of application No. 09/939,507, filed on Aug. 24, 2001, now abandoned.
(63) Continuation-in-part of application No. 10/345,825, filed on Jan. 16, 2003.
(60) Provisional application No. 60/447,922, filed on Feb. 19, 2003.

(51) Int. Cl. A61B 17/20

(52) U.S. Cl. 604/22

(57) ABSTRACT

The invention is a multi-step method for treating tissue burns. First, at least one substance that includes saline is situated substantially adjacent to the tissue. Next, at least one ultrasonic signal emitting device is affixed substantially adjacent to the at least one substance. Then, at least one ultrasonic signal emitted from the at least one ultrasonic signal emitting device is applied to the at least one substance so as to effect movement of at least a portion of the at least one substance into the tissue.

### CROSS-REFERENCE TO RELATED APPLICATIONS

This application claims priority of U.S. Patent Application Ser. No. 60/447,922, filed Feb. 19, 2003 and entitled “ULTRASONICALLY ENHANCED SALINE TREATMENT FOR BURN DAMAGED SKIN”. This application is also a continuation in part of each of: U.S. patent application Ser. No. 09/939,435, filed Aug. 24, 2001 entitled “ULTRASONICALLY ENHANCED SUBSTANCE DELIVERY METHOD”, U.S. patent application Ser. No. 09/939,506, filed Aug. 24, 2001 entitled “SUBSTANCE DELIVERY SYSTEM”, U.S. patent application Ser. No. 09/939,507, filed Aug. 24, 2001 entitled “ULTRASONICALLY ENHANCED SUBSTANCE DELIVERY SYSTEM AND DEVICE”, and U.S. patent application Ser. No. 10/345,825, filed Jan. 16, 2003 entitled “SUBSTANCE DELIVERY DEVICE”, the entire disclosures of which are each respectively hereby incorporated by reference herein as if being set forth in their respective entireties.

### FIELD OF THE INVENTION

The present invention relates generally to substance delivery methods, and more particularly to a method for enhancing transdermal substance delivery for the therapeutic treatment of burned skin.

### BACKGROUND OF THE INVENTION

In the case of burn skinned, burn victims are generally prescribed saline for the treatment of the skin tissue as a means of soothing the burn, helping to avoid infections and in aiding skin repair. Traditionally, saline is soaked onto a bandage and the bandage applied to the burn area. The saline bandage may be wrapped around the burn area and worn by the patient over a period of time, which could be as little as a few hours to as long as several weeks.

A primary problem in saline therapy is that saline will often not permeate beyond the outer skin layers and as such can prove only moderately effective.

Transdermal substance delivery systems, such as skin medicant delivery systems, may employ a medicated device or patch affixed to an exposed surface of a patient's skin. The patch allows a substance, such as a medicinal compound contained within the patch, to be absorbed into the skin layers and finally into the patient's blood stream. Transdermal skin medicant delivery often avoids pain associated with drug injections and intravenous drug administration. Transdermal skin medicant delivery may also be used to avoid gastrointestinal metabolism of administered drugs, and for providing a sustained release of an administrated drug. Transdermal skin medicant delivery may also enhance patient compliance with a drug regimen because of the relative ease of administration and the sustained release of the drug.

However, it is believed that several medicinal compounds are not suitable for conventional transdermal skin medicant delivery, since they are absorbed through the skin with difficulty, due to the molecular size of the drug or other bioadhesive properties of the drug, for example. In these cases, when transdermal skin medicant delivery is attempted, the drug may be found to merely pool on the outer surface of the skin and not permeate into the blood stream. Once such example is saline, which has been found difficult to administer by means of conventional transdermal skin medicant delivery.

Generally, conventional transdermal skin medicant delivery methods have been found suitable only for low molecular weight medications such as nitroglycerin (for alleviating angina), nicotine (for smoking cessation regimens), and estradiol (for estrogen replacement in post-menopausal women). Larger molecular medications such as saline (a polypeptide for the treatment of diabetes), erythropoietin (used to treat severe anemia) and gamma-interferon (used to boost the immune system's cancer fighting ability) are all compounds not normally effective when used with conventional transdermal skin medicant delivery methods, for example.

Methods of increasing the permeability of skin to drugs have been described, such as iontophoresis. Iontophoresis involves the application of an external electric field and topical delivery of an ionized form of drug or non-ionized drug carried with the water flux associated with ion transport (electro-osmosis). While permeation enhancement with iontophoresis has been effective, control of skin medicant delivery and irreversible skin damage are problems that may be associated with the technique.

Ultrasound has also been suggested to enhance permeability of the skin and synthetic membranes to drugs and other molecules. Ultrasound has been generally defined as mechanical pressure waves with frequencies above 20 kHz. Ultrasound signals can be generated by vibrating a piezoelectric crystal or other electromechanical element, such as through passing an alternating current through the material. The use of ultrasound to increase the permeability of the skin to drug molecules has been termed sonophoresis or phonophoresis.

However, while the use of ultrasound for skin medicant delivery has been generally suggested, results have been largely disappointing for a variety of reasons. For example, the enhancement of permeability has been relatively low. Further, it is believed that there is no consensus on the efficacy of ultrasound for increasing drug flux across the skin. While some studies report the success of sonophoresis, others have obtained negative results. Also, many conventional ultrasonic transdermal delivery systems envision a typical ultrasonic wand or sonicator as an ultrasonic applicator, not taking into account the power utilization of the transducer and the size of the device. Use of an ultrasonic transdermal delivery system in this way would be inefficient and cumbersome for a prospective user.

In general, ultrasound exposure times for permeation through human skin have conventionally been 10 minutes to 24 hours. The depth of penetration of ultrasonic energy into living soft tissue is inversely proportional to the frequency, thus high frequencies have been suggested to improve drug penetration through the skin by concentrating the effect in the outermost skin layer, the stratum corneum.

Thus, in view of the foregoing problems and/or deficiencies, there is a need for a method of treating burn victims that provides a deeper penetration of saline or other compounds suitable for the therapeutic treatment of burned skin. The present invention provides a method for safely enhancing the permeability of the skin for noninvasive skin medicant delivery in a more rapid time frame that is portable, comfortable, and easy to use.

### SUMMARY OF THE INVENTION

A method for treating tissue burns, including situating at least one substance that includes saline substantially adjacent to the tissue, affixing at least one ultrasonic signal emitting device substantially adjacent to the at least one substance, and applying at least one ultrasonic signal emitted from the at least one ultrasonic signal emitting device to the at least one substance so as to effect movement of at least a portion of the at least one substance into the tissue.

### BRIEF DESCRIPTION OF THE FIGURES

Understanding of the present invention will be facilitated by consideration of the following detailed description of the preferred embodiments of the present invention taken in conjunction with the accompanying drawings, in which like numerals refer to like parts, and:

FIG. 1 is an artist's depiction of an ultrasonic skin medicant delivery apparatus, worn by a patient upon the arm;

FIG. 2 is an artist's depiction of an ultrasonic skin medicant delivery apparatus, worn by a patient upon the abdomen;

FIG. 3 is an illustration of the structure of human skin;

FIG. 4A illustrates a cross section view of an embodiment of the transducer element of the present invention, the transducer element being a “cymbal” type transducer design;

FIG. 4B illustrates the fabrication steps to produce a “cymbal” type transducer element;

FIG. 4C illustrates a cross section view of a transducer element in a stacked “cymbal” type transducer design;

FIG. 5A illustrates dimensional characteristics of an embodiment of the present invention, including use of a polymer potting used as a resonance compatible coupling agent coating over the surface of the transducer element;

FIG. 5B illustrates the small dimensions obtained in the fabrication of a “cymbal” type transducer element;

FIG. 6 illustrates an array of transducers used to enhance sonic efficiency and to provide multiple delivery sites to the skin;

FIG. 7 illustrates an ultrasonic waveform that alternates from sawtooth to square wave is utilized;

FIG. 8 illustrates a hard transdermal drug delivery device involving a transducer coupler and a Patch-Cap;

FIG. 9 is a depiction of the connecting procedure of a hard transdermal drug delivery device involving a transducer coupler and a Patch-Cap;

FIG. 10 is a layout of the devices used in an experiment, called the U-strip Test Set Rig;

FIG. 11 illustrates the placement of a transdermal drug delivery device over an area of damaged skin;

FIG. 12A illustrates a sonogram image of skin prior to ultrasonic treatment at a depth of about 5.2 mm.;

FIG. 12B illustrates a sonogram image of skin prior to ultrasonic treatment, with saline left upon the surface of the skin, at a depth of about 5.2 mm;

FIG. 12C illustrates a sonogram image of skin after saline has been administered in conjunction with ultrasonic treatment at a depth of about 5.2 mm;

FIG. 12D illustrates a sonogram image of skin prior to ultrasonic treatment at a depth of 10.2 mm; and

FIG. 12E illustrates a sonogram image of skin after saline has been administered in conjunction with ultrasonic treatment at a depth of 10.2 mm.

### DETAILED DESCRIPTION OF THE INVENTION

It is to be understood that the figures and descriptions of the present invention have been simplified to illustrate elements that are relevant for a clear understanding of the present invention, while eliminating, for the purpose of clarity, many other elements found in typical photonic components and methods of manufacturing the same. Those of ordinary skill in the art will recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

In an exemplary embodiment of the present invention, an ultrasonic transducer device may used for enhancing the transdermal delivery of medicants for the treatment of conditions, such as disease, infection, abrasion, burned skin, general relief of pain, or any other sort of wound or ailment, for example, in a patient or user. Users may include any group of people, such as the elderly, young patients, juvenile patients, according to gender, or even patients that are at different stages of a particular disease, for example. Non-human animals may also benefit from the invention. In another embodiment, several areas of the skin may be used as transport sites. These multiple transport sites may be treated simultaneously or sequentially using multiple transducers configured into one or more transducer arrays. Multiple transducers configured in an array may be used so as to change the area of the skin used for drug absorption, i.e. the transport sites.

Transport of drug molecules across the skin surface may be accomplished using pathways associated with hair follicles and skin pores. Methods for delivery of medicants may be accomplished transdermally, transcutaneously, transmucosally, intralumenally, or within solid tissue sites, where absorption of the medication or a pharmacologically active portion thereof into the underlying or surrounding tissue is phonophoretically enhanced by the application of ultrasonic or sonic energy. Such delivery of various medicants through the skin and mucosal membranes via ultrasound may be non-invasive, and thus minimize the discomfort of the user.

According to an aspect of the present invention, the transducer device may be small, battery powered, and capable of generating an ultrasonic transmission suitable for effecting the transmission of a pharmaceutical compound from a transdermal apparatus on the skin surface of a user. Additionally, the transducer device may be worn by the patient during the course of the day through a portable device which is attached the patients body. The transducer device may also be placed directly within a drug-containing apparatus, or worn over the apparatus, and held in place by, for example, adhesive strips, body affixing straps, or other suitable methodology. In another embodiment, the transducer device may be a desktop unit used in a hospital or clinical setting. The ultrasonic transducer device may be placed directly in contact with a transdermal delivery apparatus, for the purpose of both enhancing and controlling the delivery of medications contained within the apparatus into and through the skin layer of a user, or through the skin and into the patient's blood stream.

The transdermal apparatus may be a pad, patch, bandage or wrap, and may contain any sort of substance, medication or combination of substances or medications suitable for use in the desired treatment.

Medicants for such treatment may include, by way of non-limiting example only, saline, biologically active molecules, nutritional supplements, and other therapeutic compounds, including larger pharmaceutically active compounds which are known in the art for their use in treating related conditions. The substance, or medicament, may take any conventional form, including liquids, gels, porous reservoirs, inserts, or the like, and the medication or pharmacologically active portion thereof may be intended to treat or alleviate an existing condition or prophylactically prevent or inhibit another condition of the patient. The effect of the medication may be local, such as providing for anti-tumor treatment, or may be systemic. Suitable medicaments include broad classes of compounds normally delivered through the skin and other body surfaces or into solid tissues, for the treatment of skin damage or resultant infections or severe pain to the patient as the result of burns, abrasions or wounds. Such medications may also include or incorporate anti-infectives, such as antibiotics and antiviral agents, analgesics and analgesic combinations, sedatives, pain treatments, saline and other appropriate skin treatment drugs. Both ionized and non-ionized drugs may be delivered, as well as drugs of either high or low molecular weight. Common examples of pharmaceutical or nutritional compounds which may be used directly or may be contained within a transdermal patch include, by non-limiting example: Acetaminophen, Antibiotics, Aspirin, Corticosterone, Erythromycin, Estradiol, Ibuprofen, Saline, and Steroids such as Progesterones, Estrogens, and Vitamins. Other substances, such as pharmaceutical or nutritional compounds, for nutraceutical, medicinal or pharmaceutical use may also be utilized. It may also be desirable to use the method and apparatus of the invention in conjunction with substances, such as drugs, to which the permeability of the skin is relatively low, or which may give rise to a long lag-time. Application of ultrasound as described herein may significantly reduce the lag-time involved with the transdermal administration of most drugs.

In another embodiment, the transducer device may be used for applying ultrasound to a transdermal apparatus for controlling transdermal and/or transmucosal flux rates of drugs or other molecules into the layers of burn damaged skin, into the bloodstream, or the body in general. In yet another embodiment, a Class V flextensional cymbal transducer and transducer array may be used to deliver low frequency ultrasound in a portable device at high efficiency for transdermal skin medicant delivery and therapeutic applications.

Variables such as fat content and mass of a particular patient's tissue, through which the drug will be delivered, may vary the frequency and intensity requirements used to obtain an effective dosing regimen. With this in mind, parameters of applied ultrasound may be changed to improve or control penetration include, for example, frequency, intensity, and time of exposure. Any or all three of these parameters may be modulated simultaneously in a complex fashion to increase the effect or efficiency of the ultrasound as it relates to enhancing the transdermal molecular flux rate either into or out of the human body. For example, various ultrasound frequencies, intensities, amplitudes and/or phase modulations may be used to control the magnitude of the transdermal flux to achieve a therapeutic or nutritional level. In one embodiment, the programmability and flux control may allow for optimized therapeutic delivery for a particular patient or user. The optimization may also be substance specific. The molecular structure of each medicant is different and may respond differently to ultrasound. Control of the frequency, intensity, concentration, timing of delivery, and drug regimen may be used to optimize delivery for any particular type of medicant.

According to another embodiment of the present invention, phase modulation, sinusoidal or alternating waveforms and/or frequency modulation may be used to enhance ultrasonic transdermal substance transport and increase a rate of substance delivery to a user. Ultrasound may also be combined with other techniques, such as iontophoresis, electroporation, depilatories, and/or chemical enhancers, such as surfactants, for example, to facilitate transdermal permeation. In another embodiment, acoustical energy delivered by a portable, self-powered, programmable ultrasonic transducer placed over a substance containing patch causes the substance to be transferred across the skin or other barrier or surface.

The portable ultrasonic transducer may be programmable. However, both programmable and manual operation may be utilized. Programmability may include the ability to control a quantity of drug delivered, the time interval and duration of skin medicant delivery, and the frequency and intensity of the applied control waveforms to the transducer. The portable ultrasonic transducer may also be programmed to apply acoustical energy at different times, promoting the delivery of a variable quantity of the medicinal compound over time. The portable ultrasonic applicator may also be programmed to deliver a medicinal compound to the patient continuously (sustained release) and/or intermittently (pulsed release), whichever may be deemed more appropriate to a drug maintenance and treatment regimen for a particular user. In another embodiment, the transducer device may be programmed to deliver an ultrasonic signal according to a timing circuit.

In another embodiment of the present invention, a microprocessor coupled with an Electrically Erasable Programmable Read-only Memory Device (“EEPROM”), a timer unit, and a waveform generator may be used to provide for programmability and time-dependent operation of the transdermal skin medicant delivery system. As is known in the art, this is often termed a “control unit”. Alternative devices for effecting analogous controls for implementing the present invention may also be provided.

The waveform generator may be programmed to provide a variety of waveforms, such as a sine, a square or a sawtooth waveform, for example, to control the transducer. Other waveforms as known in the art may also be utilized. The frequency of the controlled waveforms may typically be from 20 Khz to 100 KHz. The waveforms may also be sequentially interleaved to provide different waveforms for different durations and/or different amplitudes. Multiple waveforms may also be generated simultaneously. In an exemplary embodiment, a method of superpositioning or summing of waveforms may also be provided to combine, for example, square and sawtooth waveform at the transducer inputs. Waveform control outputs may be applied to a plurality of transducers simultaneously or may be divided and time phased so as to permit sequential operation of different transducer elements.

In another embodiment of the present invention, a timing generator and EEPROM may serve to store drug specific delivery scenarios in memory. For example, a basal timing sequence and a bolus timing sequence may be programmed, stored, and then retrieved and executed, thereby controlling the transducer or transducer array in a specific skin medicant delivery operation.

A pulsed or continuous mode of operation may also be selected. In addition, an electric signal, which may be directed through the skin of the patient at any time during the skin medicant delivery sequence, may also be provided. The electric signal may be programmed to be anywhere in the range of 1 to 20 Volts, for example. The electronic control unit may also be battery operated for portability and ease of use.

Encapsulation of substances and/or various compounds to be delivered may increase the permeability thereof and allow for slow time release of medication. For example, excipients may be used to improve transport through the stratum corneum and absorption into the blood stream. Several medicants or other substances may be applied using this method for local application of medication.

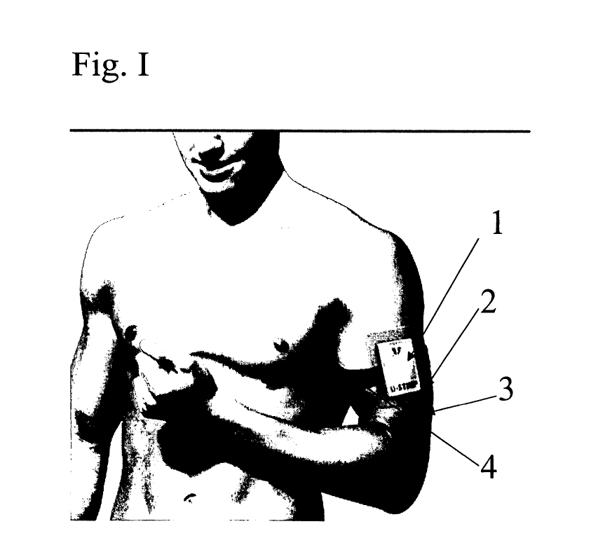

As may be seen in FIG. 1, a wearable, non-invasive, ultrasonic-transdermal skin medicant delivery system is shown. Included in this system may be an ultrasonic applicator 1 placed directly over a transdermal apparatus or patch 2. Contact between the transducer device and the transdermal patch preferably promotes efficient acoustic energy transmission. The materials and adhesives used may help maintain the intensity and power output of the ultrasonic transmission from the transducers through the transdermal patch. The applicator 1 and patch 2 may be attached to the exterior surface of a user's skin 3 by means of a strap 4, which securely holds the ultrasonic applicator 1 and patch 2 in place. Power for the ultrasonic applicator 1 may be provided by power cells (not shown), for example, which may be rechargeable and optionally located within the strap 4 itself. Alternatively, the power supply may be contained within the ultrasonic transducer device 1 or provided by any conventional external source.

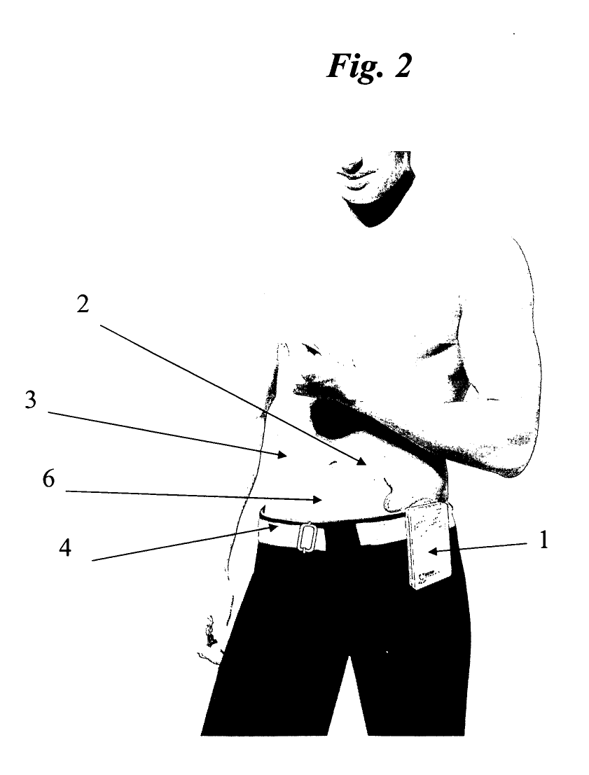

As seen in FIG. 2, device 2 may also be placed over the patient's abdomen, chest (as in the case of nitroglycerin skin medicant delivery, for example), or any other suitable part of the patient's body as determined by medical personnel administrating the drug treatment regimen. Other body placements include, but are not limited to, the neck, back and legs, for example. FIG. 2 also shows an ultrasonic applicator 1 affixed directly over the transdermal patch 2 and secured to the skin surface 3 of a user, where the transducers 4 are placed directly in contact with the transdermal patch 2, in this instance affixed to the patients abdomen 3.

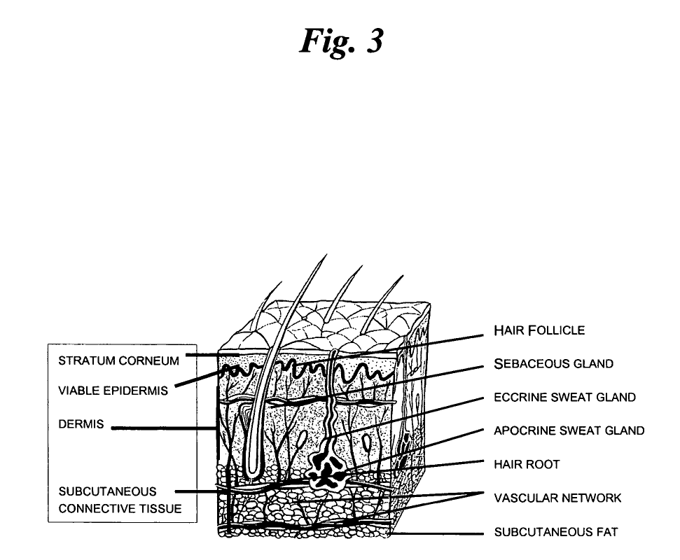

FIG. 3 illustrates the structure of human skin. Essentially there are three pathways through the skin into the bloodstream: 1) Breaching the Stratum Corneum; 2) Passing through pores in the skin; and, 3) Passing through the skin by following the hair follicle to the hair root, and from there into the vascular network located at the base of the hair root. According to an aspect of the present invention, transdermal skin medicant delivery may use pathways associated with the pore and the hair follicle system on the patient's skin. The ultrasonic frequency, intensity level and/or waveform dynamics of delivered ultrasound may, separably or in combination, be adjusted to exploit skin medicant delivery through the hair follicle pathway primarily and through the pores in the skins surface secondarily, but not directly through the stratum corneum, as it is believed the amount of energy needed for piercing the stratum corneum may be excessive and potentially damaging to fatty tissue.

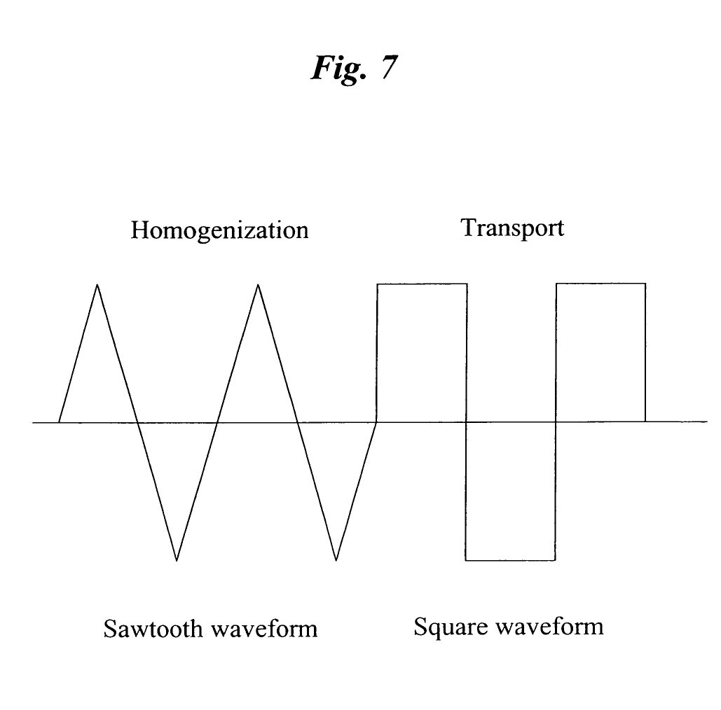

When larger sized medicants are used, or when medicants within a patch agglomerate into larger clump sizes during storage, conventional skin transport potential is reduced. To help alleviate these problems, the waveform of the ultrasonic signal may be altered, from time to time, from a sawtooth to a square waveform. Also, through the use of alternating waveforms, the amount of energy transmitted to the surface of the skin may be minimized while providing a pressure wave effect upon the skin, enhancing skin medicant delivery through the hair follicle and pore system. Referring to FIG. 7, an ultrasonic waveform that alternates from sawtooth to square wave may be used. The amplitude and intensity of such wave shaping may aid in the homogenization of a drug contained within the transdermal patch and in miniaturizing a beadlet size of the active pharmaceutical substance within the patch, as well as in drug transport through the skin. The short, peaked portion of the ultrasonic waveform in a sawtooth shape helps with drug homogenization, without imparting destructive frequencies and cavitation to the drug substance. Upon conversion to the square waveform, the ultrasonic transmission may act to massage and open the fatty tissue surrounding the hair follicle and pores. Drugs permeating from the transdermal patch are preferably in monomer form and/or reduced in droplet size, below approximately 50 Angstroms, making them more suitable in dimension to pass through the skin. The square waveform may help to “push” the drug through the pores and alongside the hair follicles, where the drug makes its way to the hair root, and directly into the bloodstream at the vascular network. Nonetheless, FIG. 7 shows an alternating waveform, where a sawtooth waveform (of relatively low average power) may be used to drive one or more transducers to homogenize a drug within a patch, leading to increased skin transport as the ultrasonic waveform stimuli switches to a square wave shape (of relatively high average power). As may be understood by those skilled in the art, the sawtooth waveform portion leads to a short period of high energy, with a short duration of pressure amplitude, leading to a vibration effect with the targeted pharmaceutical substance. This vibration is with a low heat potential and may have the effect of mixing or homogenizing the drug within the patch. Thus, smaller beadlet sizes may be made possible by the sawtooth waveform portion. In one embodiment of the present invention, the frequency and intensity of the sawtooth waveform portion of an ultrasonic signal impinging a transport site may be around 20 kHz at about 125 mW/sq. cm to about 225 mW/sq. cm, while the frequency and intensity of the square waveform portion is around 20 kHz at around 125 mW/sq.cm to around 225 mW/sq.cm. Further, each waveform may be provided for 100 milliseconds, before transitioning to the other waveform, for example. While an alternating waveform may be a preferred embodiment for the ultrasonic transmission, a conventional sinusoidal sonic or ultrasonic transmission may also be effective for the delivery of saline or other medicants through the skin. Thus, other varying waveforms having alternating average imparted powers, for example, may also be used.

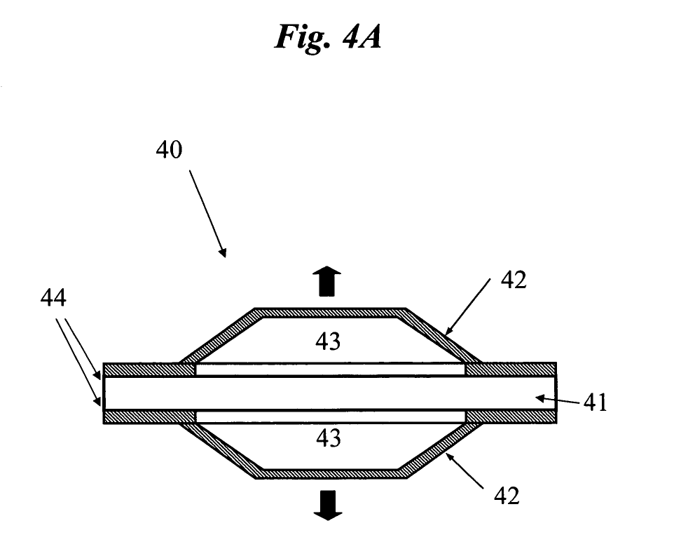

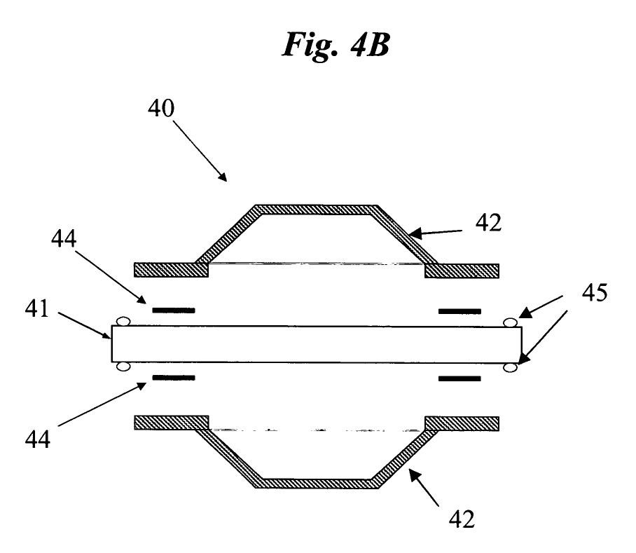

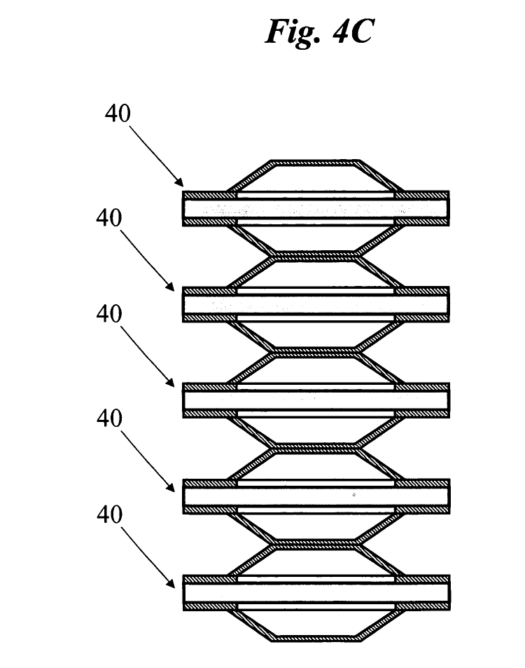

Referring to FIG. 4A, a cymbal type design of ultrasonic transducer 40 may be used in the present invention. Cymbal transducer 40 includes piezoelectric disc 41, such as PZT4, available from Piezokinetics Corp., Bellefonte, Pa., connected to two metal caps 42, which may be composed of titanium foil, for example. A hollow air space 43 between the piezoelectric disc 41 and the end caps 42 is also shown. The end caps 42 may be connected to the piezoelectric disc 41 by a non-electrically conductive adhesive 44 to form a bonded layered construction to the transducer 40. Cymbal transducers offer a thin, compact structure suitable for a portable ultrasonic skin medicant delivery system. Additionally, such a transducer may allow for the conversion of electric power to acoustically radiated power. Such a transducer design may further allow the system to be battery powered, small and lightweight.

As can be seen in FIG. 4C, a stacked cymbal type of ultrasonic transducer 40 may be used. In a stacked transducer construction, greater intensity of ultrasonic signals may be generated. U.S. Pat. No. 5,729,077, issued to Newnham et al., the entire disclosure of which is incorporated by reference herein, discloses the use of stacked transducers. Essentially, transducers are fitted atop each other to increase ultrasonic intensities while maintaining a given frequency level. A stacked transducer construction may be used to increase intensity while improving the power utilization of the transducer system.

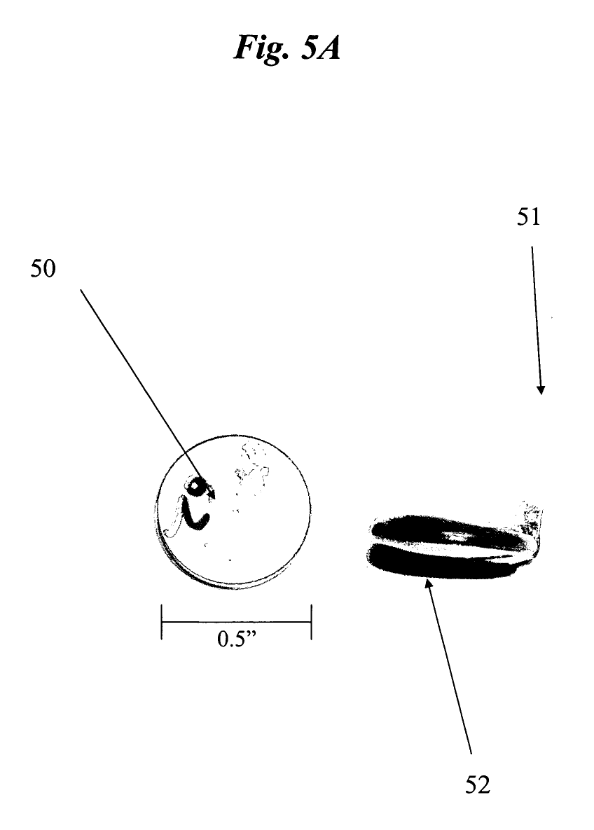

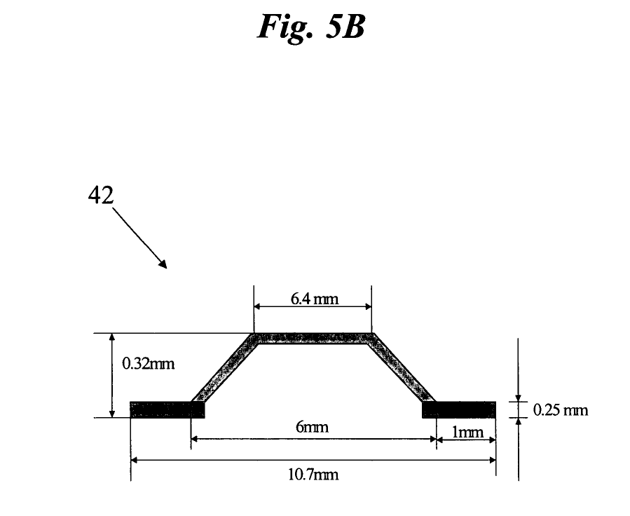

Referring to FIG. 5A, an example of the size of the transducers which may be used is shown. In one embodiment, the transducers may be around 0.5 inches in diameter. The use of such a relatively small size transducer may allow the transducers to fit within the dimensions of a transdermal patch, for example. In addition to this, the small size may allow a lower weight potential for the transducers, again aiding in the portability of the invention. The transducer element 50 may be a cymbal type construction attached to a power cable 51. The transducer element 50 may be coated in a polymer housing 52 composed of a polyurethane material, or any other material suitable for castings, coatings, and/or adhesives, such as a Uralite resin, which may typically be used to avoid short circuits between the two metallic caps 42 (See FIG. 4A), while providing acoustic coupling for the sonic transmission.

Referring to FIG. 5B, an example of the possible dimensions of a cymbal type transducer element is shown. The cymbal type transducer design may offer several advantages, such as a compact structure with a small surface area, high acoustic pressure and high acoustic intensity at low resonance frequency, a higher efficiency requiring less driving power, and the use of low resonance frequency to avoid a high cavitation threshold, i.e., the intensity required to generate air bubbles within the stratum corneum of the patient's skin tissue, for example. The cavitation threshold may be inversely proportional to the frequency applied, so the choice of a low resonance frequency of the transducer may permit a lower acoustical pressure applied to the surface of the skin, thus affecting transdermal skin medicant delivery. For a more thorough discussion regarding cymbal transducers in general, the reader may be referred to the following U.S. Patents: U.S. Pat. No. 4,999,819, (Newnham, et al), U.S. Pat. No. 5,276,657, (Newnham, et al) and U.S. Pat. No. 5,729,077, (Newnham, et al), the entire disclosures of which are hereby respectively incorporated by reference as if being set forth in their respective entireties herein.

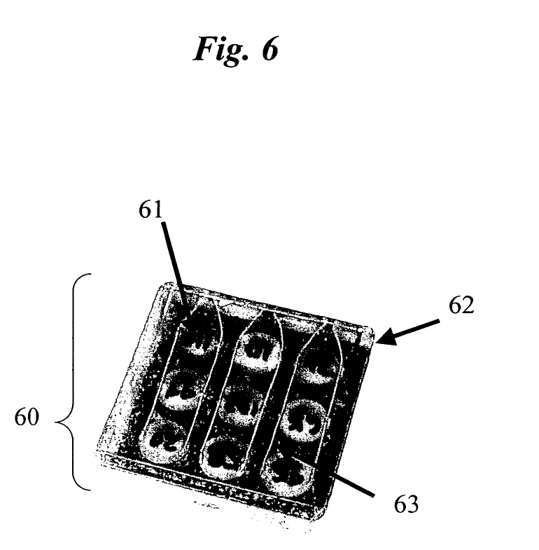

Referring now to FIG. 6, an array 60 including more than one-cymbal element 61 arranged in a pattern (or array) onto a substructure or encased within a polymer housing 62 is shown. The array may take any suitable form, such as a 2×2 array or a 3×3 array, for example. The cymbal elements 61 may be connected in parallel by a series of electrical connections 63. The array 60 may also be sealed in polymer potting material 62, such as Uralite, for example. Such an array allows for a portable, battery powered, ultrasonic transmission with sufficient power to effect skin medicant delivery via a transdermal patch.

In an embodiment of the present invention, the sonic transducer 1, as shown in FIG. 6, can transmit ultrasonic signals through multiple transducers, and therefore through multiple transport sites. The activation of one or more elements of the transducer array may be sequenced from transducer element to transducer element, optionally using different waveforms, frequency, amplitudes, and duty cycles between each transducer element, for example. This may serve to advantageously relieve the skin transport sites from continual ultrasonic stress and provide increased variability in ultrasonic skin transport energy manipulation. The transducers may also act in tandem, transmitting together.

The transducer array as shown in FIG. 6 may also provide for spreading out medicant delivery pathway sites along the skin surface by providing ultrasonic transmission from the multiple transducer elements 61 of the array acting upon the skin. The transducer elements 61 may be activated simultaneously or sequentially to transmit ultrasound through a transdermal patch and through differing multiple sites on the skin surface, for example. Additionally, the frequency, intensity and/or waveform may be altered at each transducer element 61 within the array 60. As shown previously, this variation may result in increased efficiency, enhanced power utilization and lengthening of the life span of the battery of the portable system. Also, the alternating transducer elements 61 help keep the drug homogenized within a pocket of the transdermal patch and help ensure that the skin is not overexposed to an excessive frequency of ultrasound.

An array of two or more transducers, of the cymbal type, for example, may help to push drugs through multiple skin transport sites. The transducer array may further reduce skin damage and improve an efficiency and transmitted acoustical intensity. By alternating the transducer activation sequence, it may be possible to mitigate skin exertion and to assure greater longevity for the skin transport sites.

Since ultrasound is rapidly attenuated in air, a coupling agent, preferably one having a low realizable absorption coefficient that is non-staining, non-irritating, and slow drying, may be needed to efficiently transfer the ultrasonic energy from the ultrasound transducer into the skin. When a chemical enhancer fluid or anti-irritant, or both, are employed, they may function as the coupling agent. For example, glycerin used as an anti-irritant may also function as a coupling agent. If needed, additional components may be added to the enhancer fluid to increase the efficiency of ultrasonic transduction. Ultrasound can be applied together with iontophoresis or as a pre-treatment to the application of iontophoresis, for example. Iontophoresis and/or electroporation in combination with the method and apparatus of the present invention may be used to enhance molecular transport through the skin. According to an aspect of the present invention, chemical substances, such as chemical enhancers, may be used to enhance substance transport as well.

In an exemplary embodiment of the present invention, the Cymbal Transducers may be constructed as follows: the piezoelectric ceramic material can take the form of a PZT4 disc 0.5-inch diameter, 1-mm thickness (PKI402) SD 0.500-0.000-0.040-402. This may be available from Piezo Kinetics Inc., Mill Road and Pine St., PO Box 756, Bellefontte Pa. 16823, for example. Titanium caps may be formed of Alfa Aesar, Titanium Foil, 0.25 mm thick, metal basis 5%, Item #10385, available from Alfa Aesar, A Johnson Matthey Company, 30 Bond Street, Ward Hill, Mass. 01835-8099, USA. A bonding layer material may take the form of Eccobond 45LV+catalyst 15LV, available from Emerson & Cuming, 46 Manning Road, Billerica, Mass. 01821. Low temperature soldering material suitable for use in connection with the Cymbal transducers include Indalloy Solder #1 E, 0.30″ diameter×3 ft long, which may be available from The Indium Corporation of America 1676 Lincoln Ave., Utica, N.Y. 13502. Wires may be formed from stranded wire, Gauge/AWG: 30, Catalog number (Digikey): A3047B-100-ND, Note: Maximum Temperature: 80C, Conductor Strand: 7/38, Voltage Range: 300V, Number of Conductors: 1, available from Alpha Wire Corporation. A polymer housing may be formed of Uralite FH 3550 part A/B, available from the HB Fuller Company. Ethyl Alcohol used may preferably be about 200 proof, and fine scale sand papers may be utilized.

Referring to FIG. 4B, the titanium foil may be dye cut titanium foils into several disks using a circular saw having 10.7 mm diameter, for example. One side of the disks results with edges as is conventionally understood, these edges may be removed with sand paper (fine scale). An alcohol bath can then be used to remove dust generated by sanding the disks. The disks may then be placed into a high pressure (12000 torr) shaping tool (polished side up). This step may be performed using a custom-made punch dye in order to shape the disks into the dimensions reported in FIG. 2, for example. Resulting rough edges may again be sanded, and the sanded disk again immersed in alcohol to remove dust. The disk may be wiped to remove alcohol and dust from disk. The thickness of the cap may be measured using a measuring pen. Caps having matching or substantially matching thicknesses may be matched together. This step may require a higher level of accuracy, because slight differences between the two caps may generate spurious resonance into the cymbal. The piezo disk ceramic (piezo disks) may be cleaned with alcohol. Epoxy bond may then be screen printed onto both sides of the piezo disk ceramic using a process similar to T-shirt screen-printing, for example. A ring of epoxy may be generated to glue the caps with the disks. This ring may be accurate and regular in order to avoid spurious resonances. The cymbals may then be placed on ceramic disks, and the composite structure placed into a press. This press may just keep the cymbal made in place, a tool where several cymbals are kept in place, for example. The pressed, compound structures may then be heated to approximately 70° C. for four (4) hours utilizing an oven, for example. The wires may then be soldered using a maximum temperature of about 270° C. at the electrical contact points, 4 points per piece, for example.

A transducer produced by the above procedure may be termed to be of a standard construction. To form a stacked construction transducer, two or more transducers may be placed directly atop one another as shown in FIG. 4C and fitted together. To form an array, the transducers may be generally connected in parallel, electrically within the polymer or epoxy bonding material as shown in FIG. 6, in either single element form or in a stacked construction format, for example.

A series of physical tests were conducted using a single element cymbal transducer fabricated according to the steps outlined above, using standard analysis procedures common to the ultrasonic and transducer industry. The single element transducer may produce an ultrasonic transmission within two ranges:

RANGE - A

| | |
|---|---|
| TRANSDUCER TYPE | Single element “Cymbal” design |
| FREQUENCY | 20 kHz |
| INTENSITY: LOWEST SETTING | 125 mW/sq. cm |
| DESIGN | Standard Construction |

RANGE - B

| | |
|---|---|
| TRANSDUCER TYPE | Single element “Cymbal” design |
| FREQUENCY | 20 kHz |
| INTENSITY: LOWEST SETTING | 225 mW/sq. cm |
| DESIGN | Stacked Construction |

Referring again to FIG. 6, a series of physical tests were conducted using an array of cymbal transducer elements fabricated according to the steps outlined above, using standard analysis procedures common to the ultrasonic and transducer industry. The single element transducer may produce an ultrasonic transmission within two ranges:

RANGE - A

| | |
|---|---|
| TRANSDUCER TYPE | Single element “Cymbal” design |
| FREQUENCY | 20 kHz |
| INTENSITY: LOWEST SETTING | 125 mW/sq. cm |
| DESIGN | Standard Construction using nine |
| | elements |

RANGE - B

| | |
|---|---|
| TRANSDUCER TYPE | Single element “Cymbal” design |
| FREQUENCY | 20 kHz |
| INTENSITY: LOWEST SETTING | 225 mW/sq. cm. |
| DESIGN | Stacked Construction using nine |
| | elements |

Arrays with different orientation of cymbals and with combinations of standard and stacked arrays may be used to increase efficiencies and to improve the effective delivery of drugs.

Alternating frequency outputs from the transducer array may be obtained. In tests, an array using nine single elements in a standard construction and in a stacked construction, produced frequency outputs, which were varied from around 20 kHz to about 100 kHz. Ultrasonic transmissions were found to be most uniform at the lower frequency range, i.e. around 25 kHz as compared to 40, 60 or 80 kHz. Ultrasonic transmissions were found irregular at these higher frequencies. In all cases, the transducers could be made to emit a purely sinusoidal waveform or be converted to a combination waveform consisting of sawtooth and square waves, as illustrated in FIG. 7. In these tests the ultrasonic driver circuit, e.g. the frequency generator, was set to propagate 100 milliseconds of sawtooth waveform followed immediately by 100 milliseconds of square waveform, before re-cycling back to sawtooth waveform.

The transducers, whether configured in a single element or as an array, in either a standard or stacked construction, may operate using low power. The portable nature of the final skin medicant delivery device, as depicted in the examples of FIGS. 1 and 2, may be achieved by the present system, which may be worn by the user. Accordingly, a portable power source, such as a rechargeable battery, may be used to drive the ultrasonic system. As a result, low power from standard commercially available battery sources may be used to drive the transducer, and, long duration power may be achieved, assuming at least one full day of continuous power is provided.

Tests were conducted using a nine element standard cymbal design array set to operate at 20 k Hz frequency and at varying intensity levels, powered by a standard “A” or “C” type battery. A useful power life of 25 hours was obtained at an intensity level of 200-mW/sq. cm., with continued constant usage to the transducer array. Other suitable power sources may also be used, such as “9 Volt” type batteries, for example.

Thus, transducers used may be effectively battery powered so as to drive the ultrasonic signal, and have an efficiency of the power utilization such that a low battery drain rate is exhibited, thereby extending the life of the power source.

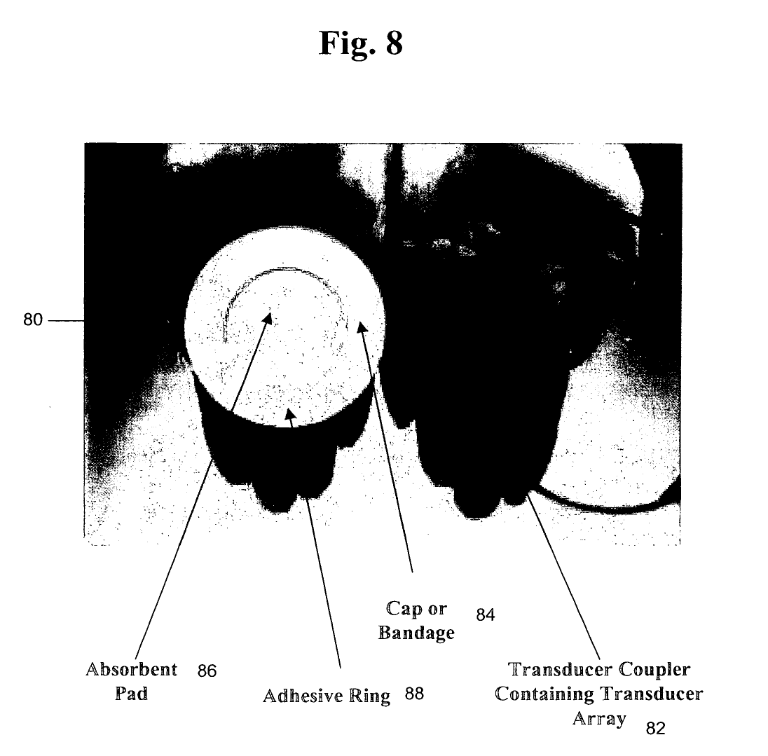

Using saline as an active skin medicant, the effect of the ultrasonic transdermal delivery discussed in connection with the present invention was tested. A four-element transducer was fabricated using four standard cymbal element transducers, as described in FIGS. 4A and 4B, in one array system, similar to the array depicted in FIG. 6. This array was set to operate at 20 k Hz frequency and at 125 mW/sq. cm intensity level per transducer element as depicted in FIGS. 5A and 5B. The transducer array was fitted with an absorbent pad composed of cotton into which 60 cc of saline was deposited. According to an aspect of the present invention, transdermal delivery device (“TDD”), as shown in FIG. 8, may have been employed. FIG. 8 illustrates a form of transdermal delivery device in which the transducer(s) 80 may be located within a coupler device 82, which snaps a transdermal patch cap or bandage 84 consisting of an absorbent pad 86 into which the medicant has been absorbed. In the patch cap an adhesive ring 88 surrounds the cap to make it easier to connect to the patients skin. If a bandage is employed the transducers may be fitted within the bandage over an absorbent material which would hold the medicant but liberate the medicant with the application of ultrasound. Common materials, such as cellulose, cotton, fabrics, synthetic fibers such as nylon or sponges, for example, may be employed to absorb the medicant.

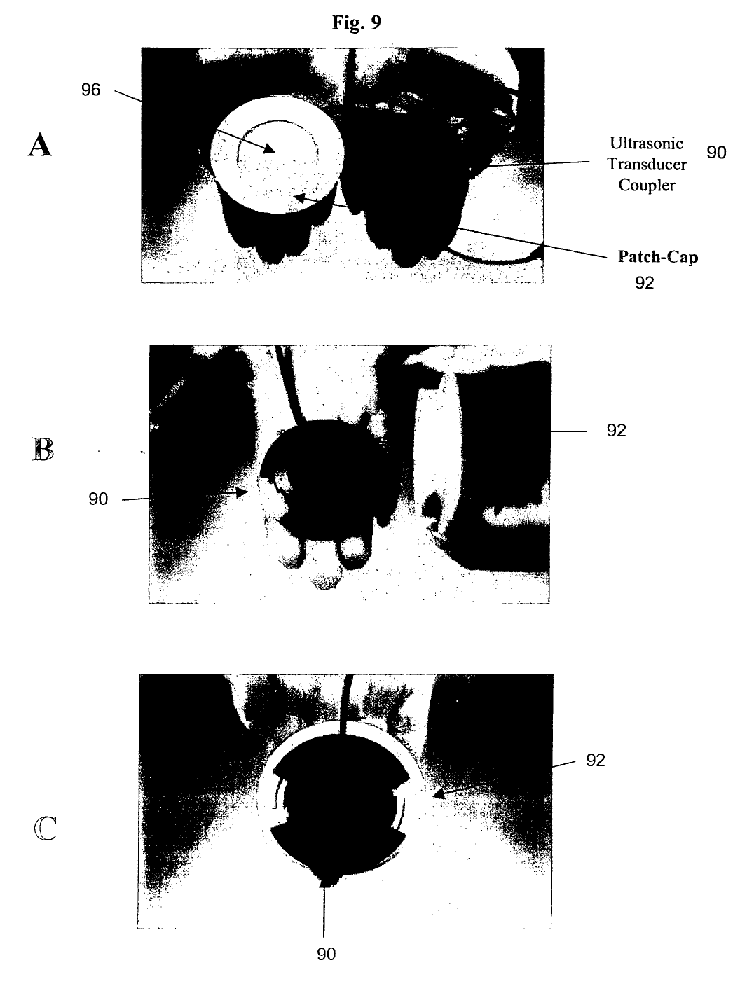

Referring to FIG. 9, an example of a preferred TDD for small area treatments on the skin is shown. FIG. 9-A shows the separate components of the TDD device, including a transducer coupler 90 and a Patch-Cap 92. FIG. 9-B shows the two components fit together using a screw on or snap on connection method. FIG. 9-C shows the completely mated TDD system, with the re-usable transducer coupler 90 on top and the Patch-cap 92 on the bottom, where connection would be made to the user's skin surface. The absorbent pad 96 may be replaceable or re-useable with multiple applications. The Patch-Cap 92 itself may also be disposable. No adhesives are used in the product, thereby reducing the chance of drug contamination.

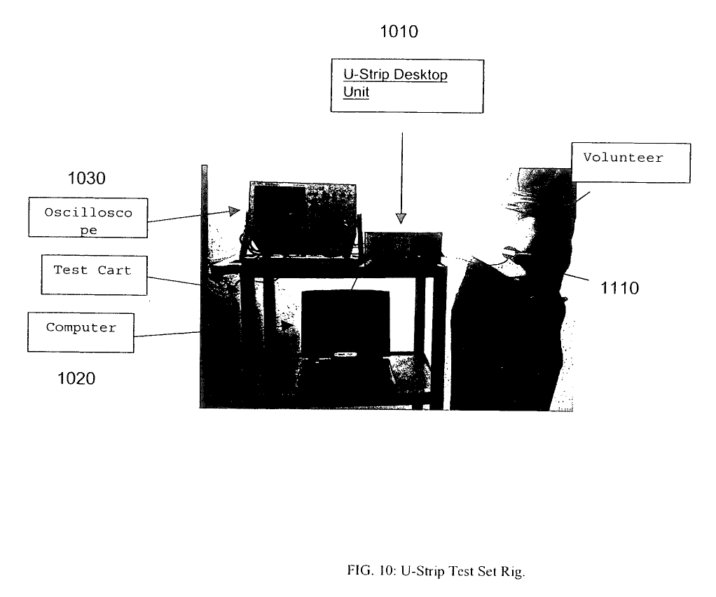

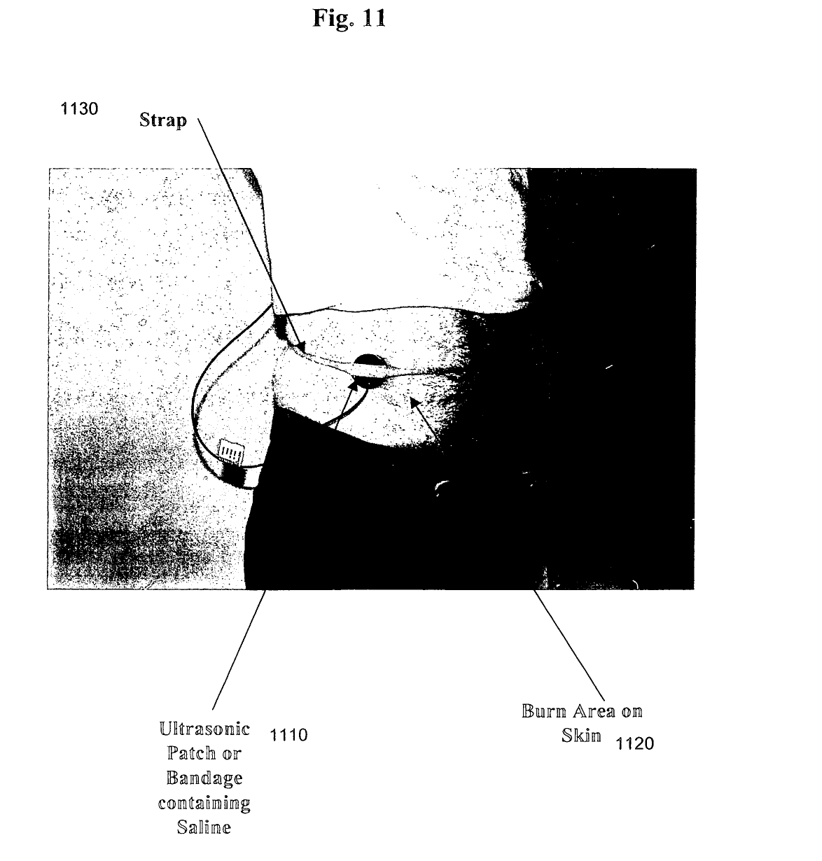

Referring to FIG. 10, a photograph of an example test rig used is shown of an experiment in which a desktop ultrasonic generator device 1010, according to the present invention, was connected to a computer 1020 for recording data and to an oscilloscope 1030 for displaying the ultrasonic frequency, intensity and sonic waveform, signal timing and other pertinent data. The transducer and patch 1110 were connected to the volunteer for this study as depicted in FIG. 11.

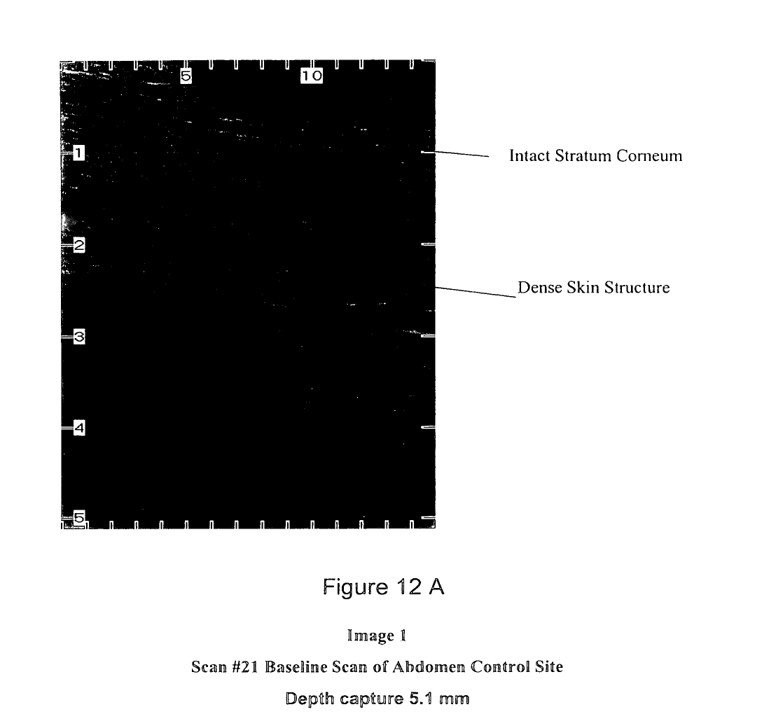

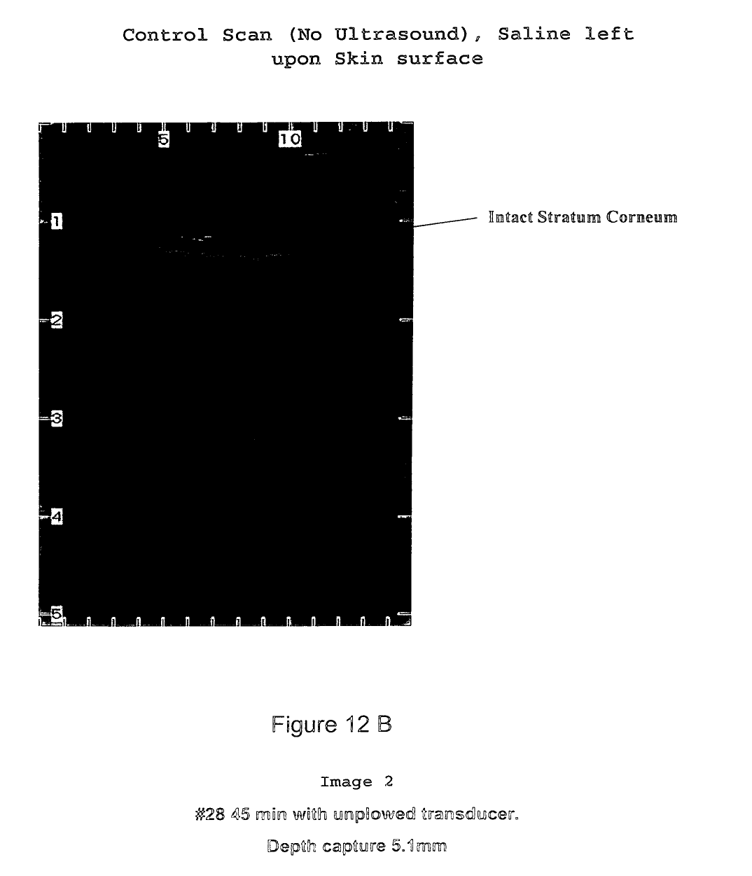

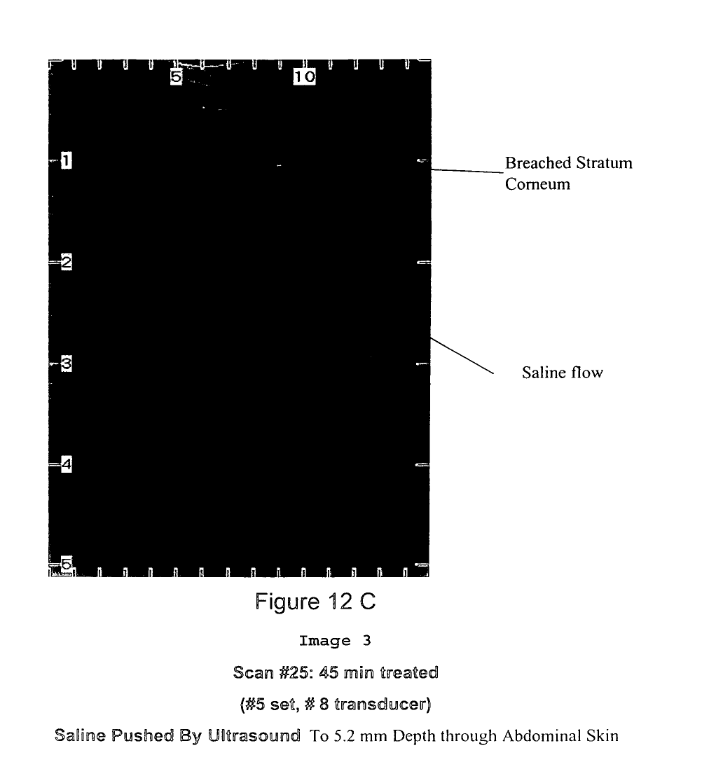

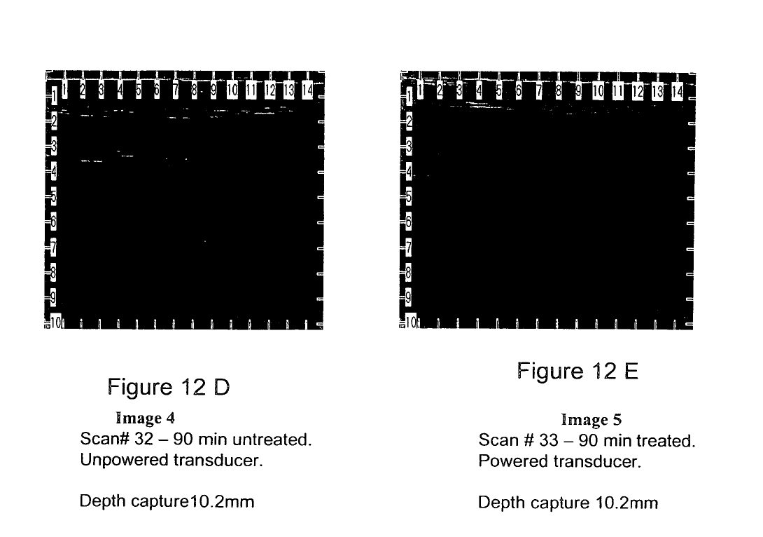

Referring to FIG. 11, an Ultrasonic patch or bandage containing saline 1110 (Transducer & Patch (TDD)) is mounted to the burn area on the skin of the abdomen 1120 and held in place by a strap 1130 over the test area. A sonar-imaging device, produced by Longport Inc., Swarthmore, Pa. was used in this experiment to provide sonar—images of the skin during the experiment. The skin surface sonar scanner was attached to the volunteer to visualize the flow of saline through the skin. Based upon this information the saline delivery program could then be set. During the study, the volunteer was not sedated. The volunteer was wired to a U-Strip desktop system and then fitted with a transducer, which held a transdermal patch mounted over the abdomen. The patch held 0.6 ml of saline. The transducer-patch 1110 was affixed to the left side of each volunteer at the locale normally taken for a hypodermic injection of saline. The site where the patch was affixed was not shaved. An alcohol wipe was used to cleanse the area prior to the placement of the saline patch. The transducer coupler contained 4 mini-transducers putting out collectively less than 0.5 watts of power. A duplicate transducer and patch were also affixed to the volunteer, but were not driven by an ultrasonic unit. An insulated electrical cable ran from the ultrasonic driver to the transducer to the volunteer. The volunteer was seated during the study, and allowed to read during testing. The duration of the first run was for 45 minutes and the second run was 90 minutes. At 0, 45 and 90 minutes, a sonoscan was made of the area under the patch placement using the sonar-imaging device. The device scanned the treated area of the skin and developed a visual representation of the flow of the saline through the skin at various depths. Testing results and data related FIGS. 12-A to 12-E are summarized in table 1, below.

TABLE 1

| | | READ ON | |
|---|---|---|---|
| IMAGE NO | DESCRIPTION | IMAGE | COMMENTS |
| 12-A | Baseline Scan At | Note green Mark | Density of skin |
| | Time Zero | at top of scan. | Structure highly |
| | Before Saline | This is the | compacted |
| | Patch is applied. | Stratum | especially at SC |
| | No Ultrasound | Corneum (SC) | layer. |
| | | before treatment | |
| 12-B | Scan after 45 | Stratum | Wet patch with |
| | minutes of wet | Corneum (SC) | Saline affixed to |
| | skin exposure to | after 45 min. Still | skin with no U/S, |
| | Saline with NO | very dense. | dampened skin. |
| | ultrasound (U/S) | | |
| 12-C | Scan after 45 | Stratum | Saline Flow |
| | minutes of | Corneum (SC) | Noticeable with |
| | exposure to | after 45 min. U/S | reduced SC |
| | ultrasound (U/S) | exposure is | presence. |
| | | fragmented | |
| 12-D | Scan after 90 | Stratum | Looking at |
| | minutes of wet | Corneum (SC) | greater depth in |
| | skin exposure to | after 90 mins. | the Dermis. Note |
| | Saline with NO | Still very dense. | higher tissue |
| | ultrasound (U/S) | | density. |
| 12-E | Scan after 90 | Stratum | At greater depth |
| | minutes of | Corneum (SC) | in the Dermis. |
| | exposure to | after 90 min. U/S | Note higher |
| | ultrasound (U/S) | exposure is | Saline Flow is |
| | | fragmented | more |
| | | | pronounced. |

The use of the Sonar scan study indicates a highly significant flow of saline was achieved through ultrasound than would have been observed using simply a wet patch laid on the skin. There was no obvious damage, irritation or marks to the volunteer's skin during the study. The volunteer observed no pain or discomfort from the application of the ultrasound. At 45 minutes the scan area was moved from 5.2 mm to 10.2 mm to determine the depth of saline permeation. It was obvious the ultrasound pushed the saline to lower skin depths, sufficient to be absorbed into the bloodstream. The pathway may suggest a subcutaneous route along the sweat pores. FIGS. 12A to 12C suggest the “breaching” of the stratum corneum (SC) where the lower depth scan was taken. FIGS. 12A and 12B show a highly defined and dense SC layer. The sonar scan suggests the SC was not ruptured but instead “softened” by the ultrasound to allow more of the saline flow through the skin. The low intensity ultrasound appears to avoid much of the detrimental effects expected with high intensity ultrasound and cavitation forces. There appears to be a priming period needed before saline begins to flow. In subsequent expanded tests the duration of skin priming by the ultrasound was reviewed. At 90 minutes the test was concluded as the saline had effectively been evacuated from the absorbent pad within the patch. In this case a cotton-based absorbent was used as the pad within the patch. This study employed continual ultrasonic transmission, employing the alternating waveform ultrasound signal associated with the U-Strip system. The use of saline in this trial indicates that many drug compounds besides saline may be delivered using the U-Strip delivery system. This test used an abdominal placement, which is significant because the site had fewer hair follicle placements and may also be a preferred delivery site for diabetics. The use of transmission in both the sonic and ultrasonic ranges may be combined to achieve optimal transport through the skin or mucosal membranes.

Those of ordinary skill in the art will recognize that many modifications and variations of the present invention may be implemented without departing from the spirit or scope of the invention. Thus, it is intended that the present invention covers the modifications and variations of this invention provided they come within the scope of the appended claims and their equivalents.

1. A method for treating tissue burns, comprising:
situating at least one substance comprising saline substantially adjacent to said tissue;
affixing at least one ultrasonic signal emitting device substantially adjacent to said at least one substance; and,
applying at least one ultrasonic signal emitted from said at least one ultrasonic signal emitting device to said at least one substance so as to effect movement of at least a portion of said at least one substance into said tissue.

2. The method of claim 1, wherein said at least one ultrasonic signal has a frequency range between about 15 kHz and about 5 MHz.

3. The method of claim 1, wherein said at least one ultrasonic signal has an intensity range between about 125-mW/sq. cm and about 225-mW/sq. cm.

4. The method of claim 1, wherein said at least one ultrasonic signal comprises about two alternating waveforms.

5. The method of claim 4, wherein said waveforms comprise a substantially square waveform portion.

6. The method of claim 4, wherein said waveforms comprise a substantially sawtooth waveform portion.

7. The method of claim 1, wherein said tissue comprises skin.

8. The method of claim 1, wherein said at least one substance is contained within an absorbent transdermal apparatus, and wherein said absorbent transdermal apparatus releases at least a portion of said at least one substance when said at least one signal is applied to said absorbent transdermal apparatus.

9. The method of claim 8, wherein said at least one ultrasonic signal emitting device is located within said absorbent transdermal apparatus.

10. The method of claim 8, wherein said absorbent transdermal apparatus is selected from the group consisting of a pad, patch, bandage, and wrap.

11. The method of claim 8, wherein said at least one ultrasonic signal emitting device comprises at least one transducer element.

12. The method of claim 11, wherein said at least one transducer element comprises an array of transducers coupled to said absorbent transdermal apparatus.

13. The method of claim 1, wherein said at least one ultrasonic signal emitting device comprises a cymbal type flat transducer.

14. The method of claim 1, wherein said at least one ultrasonic signal emitting device comprises an array of stacked transducers.
